(19) United States
(12) Patent Application Publication
(10) Pub. No.: US 2003/0211554 A1
(43) Pub. Date: Nov. 13, 2003

(54) Lysosomal pepstatin-insensitive proteinase as a novel biomarker for detecting and diagnosing breast cancer

(76) Inventors: Raju K Pullarkat, Staten Island, NY (US); Mohammed A Junaid, Dayton, NJ (US)

Correspondence Address: MORGAN & FINNEGAN, L.L.P., 345 PARK AVENUE, NEW YORK, NY, 10154, US

(21) Appl. No.: 10/221,565

(22) PCT Filed: Mar. 8, 2001
(86) PCT No.: PCT/US01/07393

(51) Int. Cl. G01N 33/574

(52) U.S. Cl. 435/7.23

(57) ABSTRACT

The present invention describes diagnostic and prognostic assays to detect in vascular and tissue samples the presence and activity of the lysosomal pepstatin-insensitive proteinase, CLN2p, which has been newly found to be associated with breast cancer and serves as a novel biomarker for breast cancer, including primary, non-primary, or metastatic breast tumors, neoplasms and carcinomas. The activity of CLN2p was discovered to be significantly elevated when measured in breast tissue samples from patients with primary breast carcinoma, compared with CLN2p levels in normal sample controls, thereby demonstrating an approximately two- to seventeen-fold higher CLN2p activity in breast tumors. These higher levels of CLN2p activity in breast tumors were positively correlated with several known breast cancer biomarkers, such as cathepsin D, estrogen receptor and progesterone receptor. The present invention thus provides CLN2p as new biomarker for use in the detection, diagnosis and prognosis of breast cancer.

This application claims benefit of provisional patent application U.S. Serial No. 60/188,861, filed Mar. 13, 2000.

The work described herein is supported in part by grants NS 30147 and CA 58183 from The National Institutes of Health.

### FIELD OF THE INVENTION

The present invention relates to a biomarker newly found to be associated with breast carcinoma and a sensitive method for detecting and diagnosing breast cancer in patients.

### BACKGROUND OF THE INVENTION

Lysosomal pepstatin-insensitive proteinase (CLN2p) is a novel mammalian enzyme discovered as a result of the identification of a gene defect in the fatal childhood neurodegenerative disorder, late-infantile neuronal ceroid lipofuscinosis (LINCL) (D. E. Sleat et al., 1997, “Association of mutations in a lysosomal protein with classical late-infantile neuronal ceroid lipofuscinosis”, Science, 277:1802-1805). The gene encoding CLN2p is called CLN2. CLN2p is an abundant lysosomal proteinase with an optimum pH of 3.5 and is extremely stable in frozen tissues or in acidified homogenates (M. A. Junaid et al., 1999, “A novel assay for lysosomal pepstatin-insensitive proteinase and its application for the diagnosis of late-infantile neuronal ceroid lipofuscinosis”, Clin. Chim. Acta, 281:169-176).

Analyses of the substrate cleavage sites have indicated that CLN2p is the same as tripeptidyl peptidase 1 (EC3.4.14.9) that cleaves tripeptides from free amino-termini bearing peptides of varying lengths of between 4-42 residues (D. J. Vines et al., 1999, “Classical late infantile neuronal ceroid lipofuscinosis fibroblasts are deficient in lysosomal tripeptidyl peptidase I”, FEBS Lett. 443:131-135; M. A. Junaid et al., 2000, “Purification and characterization of bovine brain lysosomal pepstatin-insensitive proteinase, the gene product deficient in the human late-infantile neuronal ceroid lipofuscinosis”, J. Neurochem., 74:287-294).

In carcinoma metastasis, it is believed that proteolytic enzymes, especially the lysosomal acid proteinases, can mediate tumor invasion through proteolysis of the basement membrane that separates the epithelium from the stroma. Hence, a number of laboratories have investigated the importance of lysosomal enzymes, such as cathepsin D, as prognostic markers for breast carcinomas (S. M. Thorpe et al., 1989, “Association between high concentration of Mr 52,000 cathepsin D and poor prognosis in primary human breast cancer”, Cancer Res., 49:6008-6014; A. K. Tandon et al., 1990, “Cathepsin D and prognosis in breast cancer”, N. Engl. J. Med., 322:297-302).

Some studies have reported that increased levels of cathepsin D are predictive for breast cancer recurrence (J. A. Foekens et al., 1993, “Prognostic value of PS2 and cathepsin D in 710 human primary breast tumors: multivariate analysis”, J. Clin. Oncol., 11:899-908). However, other investigators have failed to confirm this finding (M. Radvin et al., 1994, “Cathepsin D by Western blotting and immunohistochemistry: failure to confirm correlations with prognosis in node-negative breast cancer”, J. Clin. Oncol., 12 :467-474). Thus, there is disagreement in the field regarding the significance and function of cathepsin D and its relationship to breast cancer, thereby leaving a need for the discovery of other novel and reliable markers and indicators for use in the detection, diagnosis, monitoring and/or prognosis of breast cancer, including breast tumors and carcinomas.

According to the American Cancer Society (ACS), breast cancer is the second leading cause of cancer death in women. In 1999 alone, the ACS estimated that there would be more than 175,000 cases of invasive breast cancer, resulting in 43,700 deaths. The ACS also reports that early detection increases survival and treatment options. Current blood markers are employed for the diagnosis and monitoring of metastatic breast cancer and not for the early diagnosis of primary breast cancer. (K. L. Cheung et al., 2000, Cancer Treatment Reviews, 26:91-102). Thus, there is a perpetual need to be able to develop reliable breast cancer detection and. diagnosis assays for early detection and involving novel breast cancer biomarkers in an effort to save lives, ameliorate existing treatments and provide prognostic indices for breast cancer.

CLN2p is an acidic lysosomal proteinase that has been found to be nearly equal in abundance to cathepsin D in brain and other tissues. For example, cathepsin D activity in human brain (gray matter) has been reported to be 1069.6±54.4 nmole/hr/mg protein (D. E. Sleat et al., 1998, Biochem. J., 334:547-551), while human brain CLN2 proteinase activity has been reported to be 917±43 nmole/hr/mg protein (M. A. Junaid and R. K. Pullarkat, 1999, NeuroSci. Lett., 264:157-160). In addition, the activity of CLN2p is very high in tissues/organs that have actively dividing cells, such as spleen and testes. Due to its abundance, CLN2p may play a crucial role in metabolism. Table 1 presents exemplary data showing the distribution and activity of CLN2p in various rat tissues.

TABLE 1

| Tissue | CLN2p Activity (nmol/hr/mg protein) |
|---|---|
| Brain | 170 |
| Lung | 238 |
| Liver | 202 |
| Heart | 93 |
| Skeletal Muscle | 55 |
| Spleen | 1384 |
| Kidney | 487 |
| Testis | 1039 |

In some cancers, proteinases acquire extra significance due to their purported role in the metastatic process. Much effort has been expended to exploit the potential of increased proteinases during metastasis as prognostic markers to predict the disease-free or overall survival. These studies have relied on semiquantitative immunoassays to measure the levels of lysosomal acid proteases such as cathepsin D and cathepsin B (M. K. Schwartz, 1995, “Tissue cathepsins as tumor markers”, Clin. Chim. Acta, 237:67-78). However, a number of limitations are encountered in the use of semi-quantitative immunoassays. For example, in the evaluation of estrogen/progesterone receptors by immunohistochemistry, only the staining of the cell nuclei is considered positive. Unfortunately, the antigenicity of receptors may not be conserved during certain procedures used for fixation. Thus, false negative results are possible.

The present inventors have newly discovered that the CLN2p acid proteinase is present and active in tumors from patients with breast cancer, particularly, primary breast cancer, and that the amount of CLN2p activity can be correlated with other factors, such as levels of estrogen receptor, epidermal growth factor receptor, and progesterone receptor that are known to have prognostic significance for patients with breast cancer. In addition, the present invention overcomes the above mentioned limitations of semi-quantitative assays by employing a quantitative measurement of the enzyme and/or of the enzyme activity levels of CLN2p in breast cancer, i.e., in breast cancer and tumor cells.

The present invention provides the first recognition and demonstration of CLN2p as a diagnostic and/or prognostic marker for breast. carcinoma, including breast tumors and cancers, at various stages of progression. The present invention also advantageously provides the detection of CLN2p as a blood-based biomarker for breast cancer through the use of the assays described herein. The assays and results thereof according to the present invention are specific for breast cancer, since, in lung cancer, for example, CLN2p activity is similar to that seen in normal tissues (the average mean CLN2p activity, ±SD, for normal lung and lung tumor tissues are 1418±505 and 1649±200 nmol/hr/mg protein, respectively).

### DESCRIPTION OF THE DRAWINGS

FIGS. 1A and 1B depicts HPLC analyses of the CLN2p activities in normal (FIG. 1A) and tumor (FIG. 1B) tissues from a patient with primary breast carcinoma. The substrate G-F-F-L-AFC (280 μM) was incubated with 7 μg protein in 50 mM ammonium formate buffer, pH 3.5, containing 0.2 mM pepstatin-A and 0.5 mM E-64 in a final volume of 25 μl for 5 minutes at 37° C. Reactions were terminated by adding ice-cold acetone followed by centrifugation at 12,000×g for 2 min. The supernatants were evaporated to dryness, the dried residue was dissolved in acetonitrile and an aliquot was analyzed on a reversed-phase C18 HPLC column. The substrate and the product (L-AFC) were detected by a variable wavelength detector set at 340 nm.

FIG. 2 presents the scatter plot of CLN2p enzymatic activity in breast tissues from normal subjects and in tumors from patients with primary breast carcinoma. The data show proteinase activity (solid lines represent mean values) in tumor specimens from 220 patients and in normal tissues obtained from 8 non-cancer subjects undergoing reductive surgery for macromastia.

### SUMMARY OF THE INVENTION

It is an object of the present invention to provide an enzyme biomarker that has been newly discovered to be associated with breast carcinoma and active in breast carcinoma cells, including breast tumors and breast cancers. In accordance with the present invention, this enzyme is the pepstatin-insensitive lysosomal tripeptidyl peptidase CLN2p, which heretofore was disclosed only to be related to the diagnosis of a rare childhood neurodegenerative disease, late-infantile neuronal ceroid lipofuscinosis.

It is another object of the present invention to provide a method of detection and diagnosis for breast carcinoma, including breast tumors and cancers, which comprises assaying for the presence and/or the enzyme activity of CLN2p in breast carcinoma cells, or in a body tissue or vascular fluid sample, e.g., breast tissue, tissue extract, cell lysate, blood, serum, or plasma. In accordance with the present invention, comparative results are obtained by assaying normal samples, i.e., normal breast tissue, preferably from a cancer-free individual, other than the breast cancer patient. Increased CLN2p level or amount, and/or increased level of CLN2p enzyme activity, in a test sample relative to the values determined for normal controls can serve to detect and diagnose breast cancer in a patient. In addition, increased CLN2p activity (i.e., enzyme activity) in a test sample, relative to CLN2p activity in normal controls, can also serve in the prognosis of breast cancer in a patient. Further, the assays for CLN2p levels and activity of the present invention can be utilized in screening assays, for example, to screen a patient's breast tissue and blood samples for indications of cancer. In addition, the assays of the present invention can be utilized in the early diagnosis of primary breast cancer, as well as in the detection and diagnosis of non-primary breast cancer or metastatic breast cancer.

It is another object of the present invention to provide antibodies, particularly monoclonal antibodies, that are immunoreactive with CLN2p, or an immunoreactive portion thereof, and to use such antibodies in immunoassays, particularly, an enzyme linked immunosorbent assay (ELISA), to detect and measure CLN2p in breast carcinomas.

It is yet another object of the present invention to provide a sensitive and reliable enzyme assay, as well as a reliable and specific biomarker, for diagnosing breast cancer in an individual, including the stage of disease exhibited by the individual.

Yet another object of the present invention is to provide a sensitive and reliable assay, as well as a reliable and specific biomarker, for use in the prognosis of the progression of breast carcinoma in a patient, or for determining or monitoring the outcome of various treatment or therapy regimens for breast carcinoma in an individual undergoing such treatment or therapy.

Another object of the present invention is to provide a simple assay that can be performed using a vascular body fluid sample, preferably, a blood, serum, or plasma sample, taken from a patient or individual being tested for breast carcinoma or undergoing treatment for breast carcinoma.

Yet another object of the present invention is to provide diagnostic and/or prognostic assays in which CLN2p alone, or in conjunction with an assessment of the status of other cancer-associated molecules, particularly, cathepsin D levels, estrogen receptor levels, and/or progesterone receptor levels, that can be used as a diagnostic and/or prognostic biomarker, or in a panel of biomarkers, for detecting and monitoring breast carcinomas, including a variety of breast tumor and cancer types.

Further objects and advantages afforded by the present invention will be apparent from the detailed description hereinbelow.

### DETAILED DESCRIPTION OF THE INVENTION

The present invention provides assays and methods involving the pepstatin-insensitive lysosomal tripeptidyl peptidase CLN2p as a newly-discovered biomarker for the detection, diagnosis and/or prognosis of breast carcinoma. Prior to the present invention, CLN2p was disclosed and considered only to be related to the diagnosis of a rare childhood neurodegenerative disease—late-infantile neuronal ceroid lipofuscinosis.

The new finding that CLN2p is highly active and has a detectable activity in a significant number of breast carcinomas tested has been first discovered by the present inventors. According to the present invention, CLN2p and its proteinase activity have been found to be a reliable biomarker for detecting and diagnosing breast cancers, tumors and carcinomas. Because proteinases can play a prominent role in tumor progression by facilitating the detachment of tumor cells from the basement membrane and their spread to other tissues, the determination of a high level CLN2p and/or high CLN2p activity in breast tissue or vascular fluid samples is indicative of breast cancer in a patient.

That CLN2p activity levels in all but one breast tumor specimen tested were greater than the highest level observed in normal tissues (Example 2) indicates that CLN2p is advantageous as a diagnostic marker for breast cancer. In addition, high CLN2p activity levels are specific for breast cancer tissue. For example, the activity of CLN2p in lung cancer tissue was unaffected when compared with CLN2p activity in normal lung tissue.

The prognostic significance of CLN2p activity for patients who are already diagnosed with breast cancer is another aspect of the present invention which, as is appreciated by the skilled practitioner, involves direct comparisons to clinical outcomes. Published studies that have attempted to define a prognostic role of cathepsin D, another proteinase, in breast cancer have been disappointing. This may be, in part, due to different forms of cathepsin D, or to a lack of understanding of the role of cathepsin D in the stromal components of the tumor, such as infiltrating inflammatory cells (M. D. Johnson et al., 1993, Cancer Res., 53:873-877). The major secretory form of cathepsin D in estrogen receptor (ER)-positive MCF-7 cells is the precursor protein with molecular mass 52 kDa, which is proteolytically cleaved into the single polypeptide 48 kDa form, and finally processed to the two-subunit mature enzyme (S. Yonezawa et al., 1988, J. Biol. Chem., 263:6504-16511). While the 52 kDa form of cathepsin D is inactive, the mature, two subunit 48 kDa form of the enzyme is proteolytically active. By contrast, CLN2p is active only as a single polypeptide of molecular mass 46 kDa.

In further contrast to the findings for cathepsin D (B. Westley and H. A. Rochefort, 1980, Cell, 20:353-362), the present inventors have found no difference in the basal secretion or in the intracellular levels of CLN2p activity, whether or not ER-positive MCF-7 cells were previously exposed to the hormone β-estradiol. These results demonstrate that increases in levels of CLN2p and cathepsin D are controlled by independent and unrelated mechanisms in breast cancer.

While a number of earlier studies have related an increase in cathepsin D following estrogen exposure, there are indications that the cathepsin D proteinase increases even in some ER-negative breast carcinoma cell lines as well. Moreover, elevated serum levels (i.e., 10±8 pmole/ml) of cathepsin D have been reported in 60% of patients with metastatic breast cancer, while no significant elevation of cathepsin D was found in women with primary breast disease, compared with healthy women (i.e., 5±2 pmole/ml and 4±2 pmole/ml, respectively). (J. P. Brouillet et al., 1997, Cancer, 79:2132-2136).

As agreed by those in the pertinent art (e.g., G. M. Clark, 1994, Breast Cancer Res. Treat., 30:117-126), there is still a strong need for new and reliable diagnostic and prognostic biomarkers for breast cancer. In accordance with the present invention, CLN2p has many desirable properties of a clinically useful biomarker: namely, it has a high level of activity in nearly all breast tumors; it has a wide range of activity among different breast tumors; it is associated with some, but not all, previously established prognostic factors for breast cancer; the enzyme activity is stable in frozen samples that have been stored for prolonged periods, and a quantitative biochemical assay as described herein is available for accurately determining activity levels of the CLN2p proteinase in samples undergoing testing.

As described herein, CLN2 proteinase has been found by the present inventors to have high levels of activity in cells and tissue specimens from breast cancer patients, including primary breast cancer patients, and also measurable levels of CLN2p enzyme activity in plasma samples (the average mean activity, ±SD, for normal samples is 19.9±1.9 nmol/ml/h). In addition, the assays to detect CLN2p enzymatic activity in samples from breast cancer patients according to the present invention are sensitive and reliable. For example, as little as about 2 to 10 μg, preferably 5 to 10 μg, of tissue protein, or about 5 to 10 μl of plasma sample, is required. Accordingly, because the proteinase assay requires only about 2 to 10 μg of tissue protein, the diagnostic methods according to the present invention are well suited for samples that are obtained by needle biopsy.

Accordingly, one embodiment of the present invention relates to CLN2p as an assayable biomarker in cell and tissue samples obtained from individuals having breast cancer or carcinoma, including various breast neoplasms, cancers and tumors, and also including primary disease samples. In this embodiment, the present invention provides substrate specific enzyme assays that are performed on breast tissue specimens or samples, e.g. a specimen obtained from a breast biopsy or aspiration, to determine the enzyme level and activity of the CLN2p, or CLN2p functional. proteinase fragments, in the specimens or samples. (Examples 1 and 2).

Another embodiment of the present invention provides a simple blood-based assay for breast cancer, including primary breast cancer, as well as non-primary breast cancers and metastatic breast cancer. CLN2p has been found to have measurable activity in the normal plasma samples (19.9±1.9 nmol/ml/h). Moreover, the present invention has shown that increased CLN2p activity in breast tumors or cancer, including primary breast tumors and cancer, is readily detected in view of the high levels of activity of this enzyme that were newly found in samples of breast cancer tissue. Accordingly, the increased activity of CLN2p in breast cancer tissue, including primary breast cancer tissue, can be clinically associated with increased activity of this biomarker in a vascular fluid sample, such as blood, serum, or plasma, for assay purposes. The analysis of CLN2p activity in plasma provides a simple blood based biomarker for breast cancer, since increased CLN2p activity in the breast tumor may lead to increased enzyme activity in the plasma. To assay for CLN2p activity in serum or plasma, such vascular fluid derived from blood samples taken from an individual to be tested is assayed in a manner similar to that described for breast cancer tissue or cell samples. (e.g., Example 1, Materials and Methods, and Example 4).

It will be appreciated by those having skill in the art that vascular fluid, i.e., blood, serum or plasma, has a physiological (i.e. approximately neutral) pH. Because the CLN2p enzyme is most stable in an acidic environment, a blood sample to be tested is thus preferably processed within about an hour or two so as to stabilize the CLN2p prior to performing the enzyme detection and activity assays according to the present invention. For processing a blood sample, the blood sample is collected, preferably in the presence of an anticoagulant, such as EDTA (K₃EDTA) or heparin. The sample is centrifuged at low speed to separate the plasma from the cellular components, and the plasma is immediately frozen at about −20° C. until use for analysis.

In another aspect, a blood sample may be acidified immediately after collection to achieve a pH of about 3.0 to 5.5, preferably 3.5 to 5.0, and more preferably, 3.5 to 4.0, so as to stabilize the CLN2p. Preferred for lowering the pH of plasma is ammonium formate buffer, pH 3.5. Those having skill in the art will be aware that acidifying a blood sample may result in precipitation of serum albumin in the sample. Thus, removal of the precipitated albumin following acidification, or otherwise removing the non-precipitated portion of the blood sample for analysis, is preferably carried out prior to performing the CLN2p detection or activity assay on an acidified fresh blood sample according to the present invention.

In accordance with the present invention, the detection of increased levels of CLN2p or CLN2p enzymatic activity in a sample is diagnostic for breast cancer. In normal breast tissues, i.e., those obtained from individuals having no detectable breast cancer, carcinoma or tumor, or no pre-cancerous condition, CLN2p activity is in the range of from 112 to 343 nmol/hr/mg protein. However, in breast tumor or cancer tissues, (i.e., from patients having breast tumors, cancers, or carcinoma), the CLN2p activity is generally in the range of from 496 to 5787 nmol/hr/mg protein.

Comparatively, the CLN2p activity in breast tumor or cancer tissues is at least about 2 to 17 fold higher than the CLN2p activity in normal tissues. Accordingly, higher CLN2p activity in breast tissue is indicative of cancer and is directly associated with a cancerous condition. The amount or level of CLN2p in the breast tumor or cancer tissue is generally proportional to the increased activity level of the enzyme detected in the tissue. Thus, the amount or level of CLN2p is expected to be at least about 2 to 17 fold higher in breast cancer versus normal, non-cancer tissue. The normal CL2Np activity values represent those determined from tissues that have been obtained from cancer-free individuals at the time of reductive surgical procedures, rather than from tissues that appear to be normal in breast tumor or cancer patients. Such normal tissue controls were employed because apparently “normal” appearing tissues from patients with breast cancer were often found to have higher CLN2p activity. Thus, the changes in breast cancer patients can be detected by increased CLN2p activity according to this invention, even though such differences may not be detectable by means of histochemistry.

Monitoring changes in the level of the CLN2p may facilitate the diagnosis and detection of breast cancer, thereby allowing the determination of a course of treatment or therapy, or assessing outcome of a treatment or therapy for a breast cancer patient. Thus, by monitoring the level of CLN2p, an abnormal level of the enzyme, or an increase in the enzyme activity, can be indicative of a cancerous condition that can be reproducibly detected so that treatment protocols can be appropriately determined and carried out. In addition, the detection of higher levels, or sustained high levels, of CLN2p activity in a breast cancer patient, compared with normal CLN2p values may serve to indicate a poor prognosis and/or an increased chance of recurrence of breast cancer.

Another embodiment of the present invention relates to antibodies that are immunoreactive with the CLN2p enzyme, or immunogenic fragments or portions thereof, particularly for use in antibody-based assays (e.g., ELISAs) for detecting CLN2p in cell or tissue samples obtained from a breast carcinoma, tumor or cancer, or in a patient's vascular fluid sample. Both polyclonal antibodies (PAbs) and monoclonal antibodies (MAbs) specific for CLN2p, or an immunogenic fragment or portion thereof, are encompassed by the present invention. CLN2p isolated and/or purified from natural sources (e.g., M. A. Junaid et al., 2000, J. Neurochemistry, 74:287-294), or synthetically or recombinantly-produced CLN2p enzyme, or peptides thereof, may be used as an immunogen for injecting into a host animal to elicit a specific immune response and to generate anti-CLN2p antibodies. Immunogenic fragments or portions of CLN2p may be obtained by the use of proteolytic enzymes, or by the synthesis of peptides bearing epitopes that elicit an immune response and generate antibodies, using methods and protocols practiced in the art.

In another embodiment, the present invention contemplates a method of producing an antibody that is immunoreactive with CLN2p, or an immunoreactive fragment or portion thereof, comprising: immunizing an animal with CLN2p as described above as an immunogen, isolating serum from the animal, wherein the serum contains antibodies that are immunoreactive with epitopes on the CLN2p immunogen and further purifying the anti-CLN2p antibodies, if desired, using immunoglobulin purification techniques practiced in the art. Alternatively, the spleen of an immunized animal, preferably a mouse, can be used in conventionally practiced hybridoma production techniques, as described herein, and monoclonal anti-CLN2p antibodies can be produced and, if desired, isolated for use.

As used herein, “antibody” or “antibodies” refers to intact molecules as well as fragments thereof, such as Fab, F(ab)₂, and Fv, which are capable of binding an epitopic determinant of the immunogen CLN2p. As will be appreciated by those having skill in the arti the immunogen can be conjugated to a carrier protein, if desired, to increase immunogenicity, particularly, if a small peptide or CLN2p fragment is used. Commonly used carriers that are routinely chemically coupled to peptides include serum albumins, i.e., bovine, sheep, goat, or fish serum albumin; thyroglobulin; and keyhole limpet hemocyanin. The coupled immunogen-carrier is then used to immunize a recipient animal (e.g., mouse, rat, sheep, goat, or rabbit).

The term “antigenic determinant” refers to that fragment of a molecule (i.e., an epitope) that makes contact with a particular antibody. When an isolated and/or purified CLN2p is used to immunize a host animal, numerous regions of the enzyme may induce the production of antibodies which bind specifically to a given region or three-dimensional structure on the enzyme; these regions or structures are referred to as antigenic determinants or epitopes. An antigenic determinant may compete with the intact antigen (i.e., the immunogen used to elicit the immune response) for binding to an antibody.

The antibodies can be elicited in an animal host by immunization with CLN2p-derived immunogenic components, or can be formed by in vitro immunization (sensitization) of immune cells. The antibodies can also be produced in recombinant systems transformed, transfected, infected or transduced with appropriate antibody-encoding DNA. Alternatively, the antibodies can be constructed by biochemical reconstitution of purified heavy and light chains. Antibodies embraced by the present invention include hybrid antibodies, chimeric antibodies, humanized antibodies (see, for example, U.S. Pat. No. 5,585,089 to C. J. Queen et al.) and univalent antibodies. Using such antibodies, for example, CLN2p, or an immunogenic fragment or portion thereof, can be detected in a test sample by chromatography on antibody-conjugated solid-phase matrices or supports (see E. Harlow and D. Lane, 1999, Using Antibodies: A Laboratory Manual, Cold Spring Harbor Laboratory, Cold Spring Harbor, N.Y.), or by immunoassay. Preferred are antibodies that specifically recognize and bind to CLN2p, and do not recognize and bind to similar enzymes or proteins.

The antibodies can be used as an in vitro diagnostic agent to test (or screen) for the presence of CLN2p in biological samples, particularly, breast tissue samples and vascular fluid samples, in standard immunoassay protocols. Preferably, the assays which use the antibodies to. detect the presence of CLN2p in a sample involve contacting the sample with at least one of the antibodies under conditions which will allow the formation of an immunological complex between the antibody and the CLN2p antigen that may be present in the sample. The formation of an immunological complex if any, indicating the presence of CLN2p in the sample, is then detected and measured by suitable means.

The assays embraced by the present invention may be, for example, of direct format (where the labeled first antibody reacts with the antigen, e.g., CLN2p), an indirect format (where a labeled second antibody reacts with the first antibody that binds to the antigen), a competitive format (such as the addition of a labeled antigen), or a sandwich format (where both labeled and unlabelled antibodies are utilized), as well as other formats described in the art. In one such assay, the biological sample is contacted with antibodies of the present invention and a labeled second antibody is used to detect the presence of CLN2p, to which the antibodies are bound and a complex is formed between the antibody and bound CLN2p.

Such assays include, but are not limited to, radioimmunoassays (RIA), ELISA, indirect immunofluorescence assays, Western blot assays, immunohistochemical assays, chemiluminescent assays, immunoprecipitation assays, dot blot assays, slot blot assays and the like. The antibodies may be labeled or unlabeled depending on the type of assay used. Labels which may be coupled to the antibodies include those known in the art and include, but are not limited to, enzymes (e.g., horseradish peroxidase or glucose oxidase), radioisotopes, fluorogenic (e.g., fluorescein isothiocyanate (FITC), fluorescein isocyanate (FIC), 5-dimethylamine-1-napthalenesulfonyl chloride (DANSC), tetramethyl-rhodamine isothiocyanate (TRITC), lissamine, and the like), chromogenic substrates, cofactors, biotin/avidin, chemiluminescent compounds, phosphorescent compounds, colloidal gold, colored particles and magnetc particles.

As is appreciated by the skilled practitioner, in such cases in which the principal indicating group is an enzyme, additional reagents (e.g., substrates) are required for the production of a visible signal. Radioactive elements of various classes, such as ¹²⁴I, ¹²⁵I, ¹³¹I, ⁵¹Cr, (gamma ray emitters); ³²P, ³H, 35S (beta emitters), and ¹¹C, ¹⁴C, ¹⁵O, or ¹³N (positron emitters), may also be used as detectable labels. The labeled complex may be detected visually, with a spectrophotometer, or by another detector, depending on the labeling or indicating group.

Modification of the antibodies allows for coupling by any known means to carrier proteins or peptides or to known water-insoluble supports or matrices, for example, polystyrene or polyvinylchloride microtiter plates, wells, or tubes; glass tubes or glass beads; and chromatographic supports, such as paper, cellulose and cellulose derivatives, and silica. Other suitable solid supports or matrices include the following as nonlimiting examples: crosslinked dextran (Pharmacia, Piscataway, N.J.); agarose, polystyrene beads (about 1-5 microns in diameter; e.g., Abbott Laboratories, Illinois); and crosslinked polyacrylamide; nitrocellulose- or nylon-based webs, such as sheets, strips, paddles, or sticks.

One method for utilizing the antibodies according to the present invention to detect CLN2p in a breast cancer-related sample comprises an immunoassay which utilizes a solid support or matrix to which are bound antibodies that recognize all or a portion of the CLN2p, contacting the support with the sample or an aliquot of the sample and detecting the CLN2p via a radioactive or non-radioactively labeled detection molecule (i.e., an appropriate antibody) that specifically binds to the CLN2p which is bound to the anti-CLN2p antibodies adsorbed onto the solid support or matrix.

Another method for detecting CLN2p in a breast cancer-related sample comprises incubating the sample with antibodies that specifically recognize and bind to CLN2p, or an immunogenic fragment or portion thereof, under conditions that allow the antibodies to bind to the enzyme, and then determining the binding of the antibodies to the CLN2p, for example, by adding detectable antibodies that bind to a different epitope on the CLN2p and are coupled to a solid support, or by adding antibodies that bind to the antibodies already bound to CLN2p, with the added antibodies being labeled and providing a selectable marker. The added selectable antibodies, or bindable fragments thereof, may be bound to a solid support, or they many, in turn, be bound by other detectable antibodies which are bound to a support. Such immunoassay methods and variations thereof are known and practiced in the art.

As a preferred, but nonlimiting example, the present invention provides the ELISA format (Engvall et al., 1971, Immunochemistry, 8:871-4; Basic and Clinical Immunochemistry, Chapt. 22, 4th Edition, D. P. Stites et al., Lange Medical Publications, Los Altos, Calif., 1982; and D. J. Reen, 1994, Methods Mol. Biol. 32:461-6) as a specifically useful and practical antibody-based method to detect, diagnose, and/or quantify the presence of CLN2p in the breast tissue or fluid sample of an individual undergoing testing either in vivo or in vitro. A variety of antibody-based ELISA protocols are known and practiced in the art and are suitable for use in the present invention.

The present invention thus embraces a diagnostic system, particularly diagnostic kits, that are used in the ELISA format to detect CLN2p in a biological tissue/cell sample or vascular fluid. For example, a suitable diagnostic system includes, in an amount suitable for at least one assay, an anti-CLN2p specific antibody as a separately packaged immunological reagent for assaying for the presence or amount of CLN2p, or fragment thereof, in a sample. Instructions for use of the packaged reagent are also typically included. Instructions for use generally include a description of the reagent concentration, or at least one assay method parameter, such as the relative amounts of reagent and sample to be admixed, maintenance time periods for reagent/sample admixtures, temperature, buffer conditions, and the like. A preferred diagnostic system further includes a detectable label or indicating agent for signaling the formation of a complex of, for example, a CLN2p or bindable fragment thereof, and an anti-CLN2p antibody according to the present invention.

In the immunoassays for use in the present invention, an anti-CLN2p antibody, monoclonal or polyclonal, serves as an immunochemical reagent to form an immunoreaction product whose amount relates, either directly or indirectly, to the amount of CLN2p in the sample. Also contemplated are immunoassay methods for determining the amount of CLN2p, or a detectable fragment thereof, in a biological fluid sample using a CLN2p, or polypeptide fragment thereof, as a reagent to form a product whose amount relates, either directly or indirectly, to the amount of CLN2p in the sample. Such assays embrace detection, diagnostic, screening and prognostic assays.

Hybridomas that produce monoclonal antibodies against the immunogenic components of CLN2p can be produced by well-known techniques. Hybridomas can be produced by the fusion of an immortalized cell line with a B-lymphocyte that produces the desired antibody. Alternatively, non-fusion techniques for generating immortal antibody-producing cell lines are possible, and are within the purview of the present invention (see Casali et al., 1986, Science, 234:476). Immortalized cell lines are typically transformed mammalian cells, particularly myeloma cells of rodent, bovine, or human origin. Most frequently, rat or mouse myeloma cell lines are employed as a matter of convenience and availability.

Standard procedures can be used to select hybridomas, such as HAT (hypoxanthine-aminopterin-thymidine) selection. Hybridomas that secrete desired monoclonal antibodies can be selected by assaying the cells' culture medium by standard immunoassays, such as immunoblotting, ELISA, radioimmunoassay (RIA), or comparable assays. Antibodies which immunoreact with CLN2p can be recovered and isolated from the medium using standard protein purification techniques (see Tijssen, 1985, Practice and Theory of Enzyme Immunoassays, Elsevier, Amsterdam).

In another embodiment, the present invention encompasses an assay to detect CLN2p enzyme activity levels in a tissue or vascular fluid (e.g., plasma or serum) sample undergoing testing. For breast tissue samples, such assays involve the preparation of tissue extracts and homogenates using procedures practiced in the relevant art, and the supernatants therefrom. The level of CLN2p activity is measured by determining the amount of substrate hydrolysis (i.e., the amount of hydrolysis of CBZ-Arg-Gly-Phe-Phe-Leu-AFC peptide substrate to the product Leu-AFC, as described in Examples 1 and 2) that is generated by the CLN2p in the test sample. The test sample results are compared with enzyme activity levels from normal samples. High pressure liquid chromatography (HPLC) as described in Examples 1 and 2 is employed to quantify the results of the CLN2p activity analysis.

Another embodiment of the present invention relates to detection, diagnostic, or prognostic assays in which CLN2p alone, or in conjunction with an assessment of the status of other cancer-associated molecules, particularly, cathepsin D levels, estrogen receptor (ER) levels, and/or progesterone receptor (PgR) levels, can be used as diagnostic and/or prognostic biomarker(s) for detecting and monitoring breast carcinomas, including a variety of breast tumor and cancer types. CLN2p levels, assayed in conjunction with a panel of cathepsin D, ER and PgR levels, provide a diagnostic, or a prognostic, biomarker assay for breast carcinoma and possible disease outcome.

Other binding assays that are well known to the person of ordinary skill in the art are encompassed by the present invention for detecting CLN2p in a sample. Such assays include the antibody based immunoassays described above and further include numerous other binding assays, for example, in which a target protein (e.g., CLN2p) in a sample may be reacted with a binding molecule capable of specifically binding to the target protein. The binding molecule may comprise, for example, a member of a ligand-receptor pair (i.e., a pair of molecules capable of specific binding interactions), antibody-antigen, enzyme-substrate, nucleic acid-nucleic acid, protein-nucleic acid, or other specific binding pairs and members thereof as known in the art. Binding molecules may be designed so that they have enhanced affinity for the target protein. Binding molecules may be linked or coupled to a detectable label or indicator for detection by means known in the art. Means for coupling labels or indicators to binding molecules are well known in the art; commercial kits for labeling proteins and the like, are also available.

### EXAMPLES

The following examples as set forth herein are meant to illustrate and exemplify the various aspects of carrying out the present invention and are not intended to limit the invention in any way.

### Example 1

Materials and Methods

Human breast carcinoma tissues were obtained either from the National Breast Cancer Tissue Resource, Baylor College of Medicine, Houston, Tex., or from the National Cancer Institute Cooperative Human Tissue Network, Philadelphia, Pa. Tumor specimens from 220 patients with primary breast carcinoma, and normal tissues from 8 non-cancer subjects undergoing reductive surgery for macromastia, without any known signs of neoplasia or pre-cancerous condition, were used in the studies described herein. The results of the CLN2p activity in breast tumors were compared with CLN2p activity from normal breast tissue obtained as described above. “Normal-appearing” breast tissue from breast cancer patients, or from the patient whose cancerous sample is undergoing testing, are not suitable for obtaining a normal reference value, since such “normal-appearing” tissues generally had higher CLN2p activity. In contrast, unlike the significantly higher level of CLN2p activity in tumor versus normal breast tissue, the CLN2p activity in normal lung tissue was similar to the CLN2p activity detected in cancerous lung tissue.

To measure CLN2p activity in tissue sources, tissues (50-100 mg) were homogenized in 2 ml of 50 mM ammonium formate buffer, pH 3.5, containing 0.15 M NaCl and 0.1% Triton X-100 in a variable speed tissue tearor. The supernatants obtained after centrifugation at 14,000×g for 10 minutes were used for CLN2p determinations. The extracts were stored frozen at −20° C. if not used immediately.

Substrate Preparation

The preparation of the tetrapeptide substrate GFFL-7-amino4-trifluoromethyl coumarin (>99% pure) and measurement of the CLN2p activity in tissue extracts by HPLC were virtually as described by M. A. Junaid et al., 1999, Clin. Chim. Acta, 281:169-176; M. A. Junaid et al., 2000, J. Neurochem., 74:287-294; and M. A. Junaid et al., 1999, Neurosci. Lett., 264:157-160.

Synthetic peptide CBZ-Arg-Gly-Phe-Phe-Leu-aminotrifluoromethyl coumarin (AFC) was purchased from Enzyme Systems, Products, Livermore, Calif. All other chemicals were from Sigma Chemical Co., St. Louis, Mo. Reversed-phase C18 high pressure liquid chromatography (HPLC) column was purchased from Rainin Instruments Co., Woburn, Wash.

The peptide CBZ-Arg-Gly-Phe-Phe-Leu-AFC (2 mg) in 0.25 mL 50% acetonitrile was treated with 100 μg Trypsin in 100 mM Tris-HCl, pH 7.4, at room temperature until completion of the reaction. The progress of the reaction was ascertained by analyzing aliquots of the reaction mixture by reversed-phase HPLC. The product Gly-Phe-Phe-Leu-AFC that was formed was purified by reversed-phase HPLC and evaporated under nitrogen to dryness (>99% pure). These HPLC conditions are the same as described under HPLC analysis.

CLN2p Assay

An aliquot of the supernatant containing 5-10 μg protein was incubated with 7 nmol (280 μM) of the peptide substrate Gly-Phe-Phe-Leu-AFC (dissolved in 40% dimethyl formamide) in 20 mM ammonium formate buffer pH 3.5, containing 0.2 mM pepstatin-A and 0.5 mM E-64. The final volume of the assay mixture was 25 μL. Incubations were carried out at 37° C. for 5-10 min., after which the reaction was terminated by the addition of 100 μL ice-cold acetone. The supernatant obtained after centrifugation at 12,000×g for 2 min. was dried under nitrogen, resuspended in 50 μL of 60% acetonitrile, and 10 μL was used for HPLC analysis.

HPLC Analysis

The reaction products were analyzed by reversed-phase HPLC on a Microsorb-MV C18 column (0.46×10 cm, 3 μm, 100 Å) in an isocratic solvent system comprised of 55% acetonitrile and 0.1% trifluoroacetic acid at a flow rate of 0.5 mL/min. Eluates were detected by a variable wavelength ultraviolet (UV) detector at 340 nm, and the peaks were integrated using a Spectra-Physics SP 4290 integrator. The detector full-range and the chart speed were set at 0.001 and 0.5 cm/min., respectively. CLN2p activity was calculated based upon the amount of Leu-AFC formed from the area of the corresponding peaks obtained by injecting known amounts of Leu-AFC.

Protein concentrations in the extracts were determined by a modified method of Lowry et al. (O. H. Lowry et al., 1951, J. Biol. Chem., 193:265-275) using bovine serum albumin as the standard. CLN2p activity in the extracts was expressed as nmol/h/mg protein and was correlated with estrogen receptor (ER), progesterone receptor (PgR) and epidermal growth factor receptor (EGFR) levels, cathepsin D activity, S-phase fraction and DNA ploidy status.

The standard multipoint, dextran-coated charcoal assay was modified as previously described to incorporate ¹²⁵I-labeled estradiol and [³H]R5020 in a single assay, allowing the simultaneous determination of both ER and PgR (L. G. Dressler et al., 1988, Cancer, 61:420-427). Levels greater than or equal to 3 fmol/mg protein were considered to be positive for ER, and levels greater than or equal to 5 fmol/mg protein were considered to be positive for PgR.

EGFR was measured by a radioligand binding assay, using fixed concentrations of radiolabeled EGF and varying concentrations of unlabeled EGF. Malignant tissue was pulverized and homogenized prior to ultracentrifugation at approximately 108,000×g for 1 hour at 4° C. Following removal of the fat and cytosol, the membrane pellet was homogenized again and briefly centrifuged. The sample was added to labeled EGF and incubated. The separation of bound from free EGF was accomplished with a polyethyleneglycol gradient, and the bound fraction was quantified on a gamma counter. Following subtraction of nonspecific binding, the data were analyzed via Scatchard analysis and reported in fmol/mg of membrane protein. Levels greater than or equal to 10 fmol/mg protein were considered to be positive.

An immunoradiometric (IRMA) variant of the double antibody technique was used to measure cathepsin D levels in tumor cytosols. Cytosols were prepared in 10 mM Tris buffer containing 1.5 mM EDTA, 5 mM dithiothreitol and 10 mM molybdate. Cytosol protein concentration was adjusted to approximately 1-2 mg/mL prior to a 60-fold dilution. Three reference cytosols prepared from pooled disaggregated breast tumor tissue were included in each assay, together with a control provided by the manufacturer. The immunoradiometric assay (CIS-US, Inc., Bedford, Mass.) employed the D7E3 monoclonal antibody as the capture antibody and the radiolabeled M1G8 monoclonal antibody as the detection antibody. Bound radioactivity was directly proportional to the level of cathepsin D in the specimen. Cathepsin levels greater than 50 pmol/mg cytosol protein were considered high.

DNA analyses were performed on breast tumor or cancer cells. DNA ploidy and S-phase fraction were determined by flow cytometry as described previously (L. G. Dressler et al., 1988, Cancer, 61:420-427 and C. R. Wenger et al., 1993, Breast Cancer Res. Treat, 28:9-20). Ploidy refers to the ratio of the amount of DNA in a tumor to the amount of DNA in normal diploid cells. S-phase by flow cytometry measures all cells that are actively synthesizing DNA. S-phase fractions were estimated using the MODFIT program (Verity Software House, Inc., Topsham, Me.), with single-cut debris stripping and single trapezoids for the S-phase components. S-phase fractions greater than or equal to 6.7 for diploid tumors and greater than or equal to 11.0 for aneuploid tumors were considered high.

Statistical Analyses

CLN2p activities in normal and breast carcinoma tissues were expressed as mean ±SD and compared by a two-sample Student's t test at the 5% level of significance. Associations between CLN2p activity and other biomarkers for breast carcinoma were described by Spearman rank correlations (rₛ) or Wilcoxon rank sum tests.

### Example 2

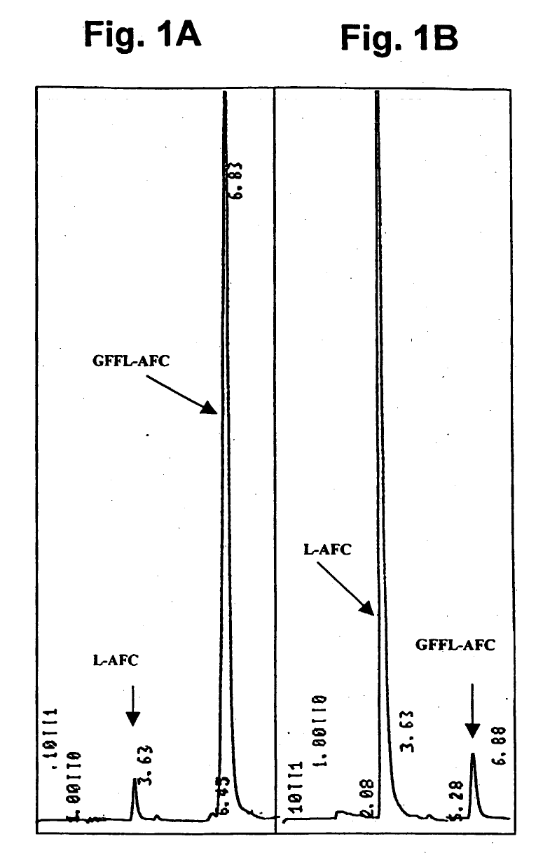

HPLC analyses of CLN2p activity in breast tumor tissue from a patient with breast carcinoma and in normal non-cancerous breast tissue were performed. The results are shown in FIGS. 1A and 1B. In the normal tissue, less than 5% of the substrate was hydrolyzed to L-AFC, while over 90% of the substrate was hydrolyzed in the cancer tissue, when the same amount of protein was incubated. In the patient samples presented in FIGS. 1A and 1B, these values correspond to CLN2p specific activity of about 300 and 3000 nmol/h/mg protein for the normal and the tumor tissues, respectively.

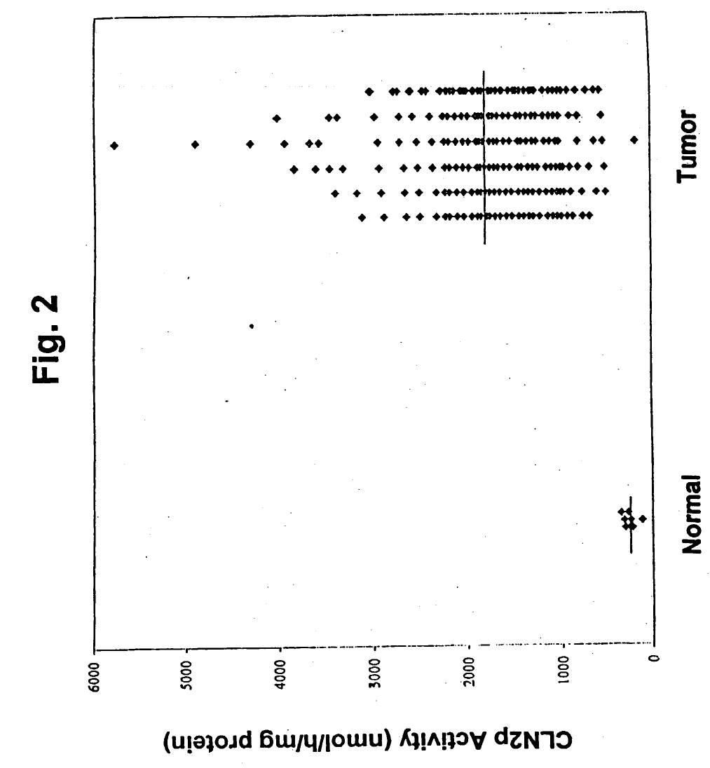

FIG. 2 shows the scatter plot of CLN2p activity in breast tissues from normal subjects and in tumors from patients with primary breast carcinoma. The levels of CLN2p ranged from 112-343 nmol/h/mg protein for normal tissues (n=8) and 164-5787 nmol/h/mg protein for tumor tissues (n=220).

The results indicate that breast tumors have at least a seven-fold higher CLN2p activity (1813±834, mean ±S.D. P<0.001) when compared with tissues from normal subjects (251±66, mean ±S.D.). All but 1 sample out of the total 220 tumor tissues analyzed had CLN2p activity greater than the highest normal value determined. This suggests that CLN2p activity is a clinically useful diagnostic test for breast carcinoma. It is not known why one of the tumor tissues possessed such low CLN2p activity. Interestingly, this one particular sample (with CLN2p activity of 164 nmol/hr/mg protein) appeared to be aberrant in other ways in that it also had a lower cathepsin D value, negative ER, PgR and EGFR levels, diploid DNA ploidy status and low S-phase fraction. Without wishing to be bound by theory, or limited by one atypical sample, simple biological variation or mutation may be attributed to the aberrant values obtained for the single anomalous patient sample showing a low CLN2p value.

### Example 3

CLN2p activity was compared with other therapeutic biomarkers measured in the breast cancer specimens (see Example 1). Associations with these factors expressed as dichotomous variables are shown in Table 2.

Test descriptions for Estrogen Receptor (ER)/Progesterone Receptor (PgR) indicate that the presence and levels of hormone receptors in tumor cells (e.g., breast tumor cells) are strongly correlated with response to hormonal therapy and clinical outcome. (H. Battifora, 1994 Appl. Immunohistochem., 2(3):143-145; S. M. Veronese et al., 1995, Appl. Immunohistochem., b3:85-90; I. deMascarel et al., 1995, Appl. Immunohistochem., 3:222-231; L. P. Pertshuk et al., 1996, Cancer, 77:2514-2519; N. Weidner, 1996, Calif. Seminars in Pathology, 10-14; C. Cohen et al., 1997, Abstr. Annual Meeting of the USCAP, 181A; C. T. Taylor, 1996, Cancer, 77(12):2419-2422). The overexpression of Epidermal Growth Factor Receptor (EGFR) is associated with poor prognosis. (N. Weidner et al., 1994, Breast Cancer Res. Treatment, 29:97-107; J. Baselga et al., 1994, Breast Cancer Res. Treatment, 29:127-138).

TABLE 2
Comparison of CLN2p activity with different levels of other
biomarkers in 200 samples from patients with primary
breast carcinoma

| | | | CLN2p activity | |
|---|---|---|---|---|
| | | | nmol/hr/mg | |
| | | | protein | Wilcoxon |
| Marker | Status | Number | (Mean ± S.D.) | p-value |
| Cathepsin D | low | 133 | 1705 ± 784 | <0.0001 |
| | high | 67 | 2157 ± 824 | |
| Estrogen receptor | − | 39 | 1400 ± 594 | <0.0001 |
| (ER) | + | 161 | 1967 ± 835 | |
| Progesterone | − | 90 | 1516 ± 586 | <0.0001 |
| receptor (PgR) | + | 110 | 2135 ± 885 | |
| Epidermal Growth | − | 154 | 1917 ± 876 | 0.16 |
| Factor Receptor | + | 46 | 1654 ± 583 | |
| (EGFR) | | | | |
| DNA ploidy | Diploid | 97 | 1839 ± 873 | 0.41 |
| | Aneuploid | 103 | 1874 ± 778 | |
| S-phase fraction | Low | 117 | 1918 ± 886 | 0.33 |
| | High | 83 | 1769 ± 723 | |

CLN2p activity was positively correlated with cathepsin D, ER and PgR (P<0.0001). No correlations, either positive or negative, were observed with EGFR, DNA ploidy status or S-phase fraction. Similar results were observed when the prognostic biomarker measurements were expressed as continuous variables (Table 3).

TABLE 3
Comparison of CLN2p activity with other biomarkers in 200
samples from patients with primary breast carcinoma

| | | Correlation | |
|---|---|---|---|
| | Values | coefficient, | |
| Variable | (Mean ± S.D.) | (rₛ) | p-value |
| Cathepsin D | 47.4 ± 32.5 | 0.45 | 0.0001 |
| Estrogen receptor | 121 ± 165 | 0.31 | 0.0001 |
| Progesterone | 191 ± 319 | 0.43 | 0.0001 |
| receptor | | | |
| EGFR | 33.1 ± 212 | −0.11 | 0.12 |
| S-Phase fraction | 9.1 ± 6.3 | −0.04 | 0.58 |

As seen from Table 3 above, CLN2p activity was significantly correlated with cathepsin D (rₛ=0.45, p<0.0001), as determined by immunohistochemical staining of cytoplasm and stroma of tumor cells; ER (rₛ=0.31, p<0.0001) and PgR levels (rₛ=0.43, p<0.0001), as determined by immuno-histochemical staining of tumor cell nuclei. However, no significant correlations were observed with EGFR levels or with S-phase fraction. Multivariate regression analyses, using both continuous or dichotomous representations, revealed that cathepsin D and PgR were the strongest predictors for CLN2p activity in the breast cancer tissues (r²=0.24 and 0.20, respectively). Collectively, the results from Example 3 suggest that CLN2p alone, or in conjunction with cathepsin D, ER and/or PgR status, can be an attractive prognostic biomarker assay for breast carcinomas, including breast cancers and tumors.

This example demonstrates that an analysis of the levels of CLN2p activity, cathepsin D activity, and ER and/or PgR levels may be employed as reliable biomarkers in a panel of biomarkers for the detection and/or prognosis of breast cancer to provide a reliable indication of a patient's breast cancer disease or condition outcome.

### Example 4

This example describes the use of an assay method according to the present invention to determine CLN2p in blood samples, particularly, plasma samples.

For plasma samples, freshly collected heparinized blood was centrifuged at low speed (˜1000×g) to separate the plasma component from the cellular component. The plasma was stored frozen at −20° C. if it was not used immediately for analysis. Prior to carrying out the assay, the plasma samples were centrifuged at 12,000×g for 5 minutes to remove any contaminating platelets. For CLN2p activity determinations, 5 μl of plasma was incubated with 280 μM of the peptide substrate Gly-Phe-Phe-Leu-AFC in 20 mM ammonium formate buffer, pH 3.5 containing 0.2 mM pepstatin-A and 0.5 mM E-64 (inhibitor of enzymes having active thiol groups, Sigma). The final volume of the assay mixture was 25 μl. Incubations were carried out at 37° C. for 10 minutes, after which the reaction was terminated by the addition of 100 μl of ice-cold acetone. The supernatant obtained after centrifugation at 12,000×g for 2 minutes was dried under nitrogen, resuspended in 50 μl of 60% acetonitrile, and 25 μl was used for HPLC analysis as described in Example 1.

The results from 8 normal plasma samples obtained using the above protocol showed that CLN2p activity in these samples was 19.9±1.9 nmol/hr/ml plasma. A plasma sample obtained from a breast cancer patient using the protocol above and assayed for CL2Np activity as described herein showed CLN2p activity that was about 10-fold higher than that of the normal plasma controls as presented in Table 4. Thus, plasma provides a suitable vascular fluid source from which to obtain CLN2p activity data and results.

TABLE 4
CLN2 proteinase activities in plasma

| | Proteinase Activity |
|---|---|
| Sample | (nmol/hr/ml) |
| Normal | 19.9 ± 1.9* (17.0-22.3) |
| Primary breast cancer | 190.8 ± 81.8 (138-293.6) |

*Mean ± standard deviation

The contents of all patents, patent applications, published articles, books, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A method of diagnosing breast cancer, comprising
(a) contacting a breast tissue or vascular fluid sample undergoing testing with an anti-CLN2p antibody under conditions that allow the formation of a complex between the antibody and CLN2p in the sample; and
(b) detecting the complex; wherein an increased amount of CLN2p in the sample undergoing testing versus the amount of CLN2p in a normal, cancer-free control sample diagnoses breast cancer.

2. The method according to claim 1, wherein the CLN2p is contacted with the antibody in an immunoassay selected from the group consisting of radioimmunoassay, Western blot assay, immunofluorescent assay, enzyme immunoassay, immunoprecipitation assay, chemiluminescent assay, immunohistochemical assay, dot blot assay and slot blot assay.

3. The method according to claim 1, wherein the antibody is a monoclonal antibody.

4. The method according to claim 1, wherein the antibody is a polyclonal antibody.

5. The method according to claim 2, wherein the immunoassay is an enzyme linked immunosorbent assay or ELISA.

6. The method according to claim 1, wherein the anti-CLN2p antibody is detectably labeled.

7. The method according to claim 6, wherein the complex is detected by a label or indicator selected from the group consisting of immunofluorescent label, chemiluminescent label, phosphorescent label, enzyme label, radiolabel, avidin/biotin, colloidal gold particles, colored particles and magnetic particles.

8. A method of diagnosing breast cancer in a patient, comprising:
(a) contacting a solid matrix having antibodies immunoreactive with CLN2p immobilized thereto with a patient's breast tissue sample or a vascular body fluid sample to allow CLN2p in the sample to bind to the immobilized antibodies;
(b) removing unbound sample;
(c) contacting the solid matrix with a labeled antibody specific for bound CLN2p; and
(d) quantifying the amount of CLN2p in the patient's sample and comparing the amount obtained in (d) with the CLN2p amount in a normal, non-cancer control, wherein an increased amount of CLN2p in the patient's sample diagnoses breast cancer in the patient.

9. The method according to claim 8, wherein the sample is breast carcinoma, tumor or cancer tissue.

10. The method according to claim 8, wherein the sample is blood, serum or plasma.

11. The method according to claim 8, wherein the label is selected from the group consisting of enzyme label, fluorescent label, chemiluminescent label, phosphorescent label, radioisotope label, colloidal gold, colored particles, magnetic particles and biotin/avidin.

12. The method according to claim 8, wherein the antibody is polyclonal or monoclonal.

13. The method according to claim 8, wherein the amount of CLN2p in the patient's sample is at least 2-fold greater than the CLN2p amount in the normal control.

14. A method of diagnosing breast cancer in an individual undergoing testing, comprising: measuring the level of enzyme activity of CLN2p in a breast tissue or vascular fluid test sample; and comparing the CLN2p activity level measured in the test sample with the level of CLN2p activity in a normal sample control; wherein an increase in the activity of CLN2p in the test sample relative to the control sample diagnoses breast cancer in the individual.

15. The method according to claim 14, wherein the vascular fluid test sample is selected from the group consisting of blood, serum and plasma.

16. The method according to claim 14, wherein the vascular fluid test sample is acidified prior to measuring the CLN2p activity.

17. The method according to claim 16, wherein the test sample is acidified to a pH of between about 3 to about 5.5.

18. The method according to claim 14, wherein the CLN2p activity in the test sample is at least about 2 to 17-fold greater than the CLN2p activity in the normal sample control.

19. The method according to claim 14, wherein CLN2p activity is measured by the amount of enzyme substrate hydrolyzed by the CLN2p in test samples versus normal control samples.

20. The method according to claim 19, wherein the substrate is hydrolyzed to Leucine-aminotrifluoromethyl coumarin (L-AFC) product and the remaining substrate and L-AFC product are detected by high pressure liquid chromatography.

21. A method of diagnosing breast cancer in a vascular fluid sample from an individual undergoing testing, comprising:
(a) assaying an aliquot of the vascular fluid sample to determine CLN2p activity; and
(b) comparing the CLN2p activity in the test sample with CLN2p activity in a normal, noncancerous sample, wherein an increase in the test sample CLN2p activity relative to the normal sample CLN2p activity diagnoses breast cancer.

22. The method according to claim 21, wherein the vascular fluid sample is selected from the group consisting of blood, serum and plasma.

23. The method according to claim 22, wherein the vascular fluid sample is plasma.

24. The method according to claim 21, wherein the CLN2p activity in the test sample is at least about 2 to 17 fold higher than the CLN2p activity in the normal sample.

25. A method of diagnosing breast cancer by determining enzyme levels of CLN2p in a vascular fluid sample from an individual undergoing testing, comprising:
a) acidifying the vascular fluid sample to a pH of about 3.0 to about 5.5;
b) assaying the acidified sample for CLN2p activity; and
c) comparing the CLN2p activity in the individual's test sample with CLN2p activity in a normal, noncancerous sample, wherein an increase in CLN2p activity in the test sample relative to CLN2p activity in the normal sample CLN2p diagnoses breast cancer.

26. The method according to claim 25, wherein the vascular fluid sample is blood, serum or plasma.

27. The method according to claim 25, wherein the vascular fluid sample is plasma.

28. An assay method for detecting or diagnosing breast cancer, comprising: determining CLN2 proteinase levels in a breast tissue or vascular fluid sample from a patient undergoing testing for cancer, and comparing the levels of CLN2 proteinase in the patient's sample with the levels of CLN2 proteinase in a normal sample, wherein increased levels of CLN2p in the patient's test sample compared with levels of CLN2 proteinase in the normal sample allows the detection or diagnosis of breast cancer in the patient.

29. The assay method according to claim 28, wherein the level of CLN2 proteinase in the patient's test sample is compared with cathepsin D level; estrogen receptor level; and progesterone receptor level in the same patient's sample; and further wherein increased levels of CLN2 proteinase, cathepsin D, estrogen receptor, and/or progesterone receptor in the test sample compared with CLN2 proteinase, cathepsin D, estrogen receptor, and/or progesterone receptor levels in a normal non-cancer sample control allow the diagnosis of breast cancer.

30. The assay method according to claim 28, wherein the vascular fluid sample is selected from the group consisting of blood, serum and plasma.

31. The assay method according to claim 30, wherein the vascular fluid sample is plasma.

32. The method according to claim 28, wherein the level of CLN2 proteinase is detected using high pressure liquid chromatography.

33. The method according to any one of claims 1, 8, 14, 21, 25 and 28, wherein the breast cancer detected or diagnosed is selected from the group consisting of primary breast cancer, non-primary breast cancer and metastatic breast cancer.

34. A diagnostic system to detect CLN2p in a biological sample or vascular fluid, comprising: a) an anti-CLN2p specific antibody in an amount suitable for at least one assay for detecting the presence or amount of CLN2p, or fragment thereof, in a sample; b) instructions for use comprising a description of reagent concentration and/or at least one assay parameter selected from the group consisting of: relative amounts of reagent and sample to be admixed, maintenance time periods for reagent and sample admixtures, temperature and buffer conditions; and, optionally, c) a detectable label or indicating agent for signaling the formation of a complex of CLN2p, or bindable fragment thereof in the sample and the anti-CLN2p antibody.
